(19) United States
(12) Patent Application Publication
(10) Pub. No.: US 2025/0148560 A1
(43) Pub. Date: May 8, 2025

(54) METHOD OF GENERATING PERSONALIZED LEARNING PATH BASED ON MULTI-COURSE KNOWLEDGE GRAPH MERGING AND SYSTEM THEREOF

(71) Applicant: Xuzhou Medical University, Xuzhou (CN)

(72) Inventors: Xiang Wu, Xuzhou (CN); Shiting Zhang, Xuzhou (CN); Lili Wang, Xuzhou (CN); Yongting Zhang, Xuzhou (CN); Zehua Yu, Xuzhou (CN); Huanhuan Wang, Xuzhou (CN)

(21) Appl. No.: 19/013,276

(22) Filed: Jan. 8, 2025

(30) Foreign Application Priority Data
Feb. 28, 2024 (CN) 202410220313.1

(51) Int. Cl. G06Q 50/20; G16H 40/67

(52) U.S. Cl. CPC G06Q 50/2053; G16H 40/67

(57) ABSTRACT

Provided is a method of generating a personalized learning path based on multi-course knowledge graph merging and a system thereof, in particular to the technical field of personalized learning path generation. The method includes: constructing a personalized knowledge base to obtain a personalized knowledge graph of all courses that students need to learn in the current academic year; using an entity alignment technology to realize the personalized knowledge graph merging of all courses, and automatically generating a personalized knowledge graph of multi-course merging; generating a subsequent learning path according to a current learning progress of users and the personalized knowledge graph of multi-course merging; and evaluating the generated learning path in terms of link prediction accuracy.

### TECHNICAL FIELD

The present disclosure relates to the technical field of personalized learn path generation, and in particular to a method of generating a personalized learning path based on multi-course knowledge graph merging and a system thereof.

### BACKGROUND

Personalized teaching is a teaching method that pays attention to individual differences and demands of students. This teaching method puts learners at the core of the teaching process and focuses on providing students with personalized learning support and resources according to the characteristics, the ability level and the interests of students. The goal of personalized teaching is to promote the all-round development of students. Students can study at their own pace and select the learning content according to their own interests.

At present, there are many personalized teaching methods to improve the learning effect and interests of students and promote the all-round development of students by understanding the characteristics, the ability level and the interests of students. However, most of the methods focus on the teaching design of single-subject teaching materials without considering the multi-subject teaching linkage process.

### SUMMARY

In order to solve the problems that the existing personalized knowledge graph has a unique teaching material and cannot automatically generate a multi-course learning path.

The present disclosure provides the following technical scheme. A method of generating a personalized learning path based on multi-course knowledge graph merging, wherein the method includes constructing a personalized knowledge base to obtain the personalized knowledge graph of all courses that students need to learn in the current academic year, further including the following steps:
Step 1: using an entity alignment technology to realize the personalized knowledge graph merging of all courses, and automatically generating a personalized knowledge graph of multi-course merging by establishing an index table;
Step 2, generating a subsequent learning path according to a current learning progress of users and the personalized knowledge graph of multi-course merging;
Step 3: evaluating the generated learning path in terms of link prediction accuracy.

Preferably, a step of constructing a personalized knowledge base is as follows.
A, physiological data signals of students, including electroencephalogram signals, electrocardiogram signals and video signals, are collected through a head-mounted electroencephalogram device, a patch electrocardiogram device and a suspended monitoring device.
B, key features are extracted from the collected physiological data signals to obtain key data features of students, including electroencephalogram, electrocardiogram, body temperature, posture and facial expression features.
C, a multimodal physiological data merging mining method is designed to perform data mining and predict learning preferences of students.

The multimodal physiological data merging mining method is designed as follows.

The electroencephalogram key features, the electrocardiogram key features and the video key features are trained, respectively. The optimal parameter models of matching features are configured for different key features. The results output by the model of the electroencephalogram key features, the electrocardiogram key features and the video key features are input into a Convolutional Neural Network (CNN) model for training as training features. The final prediction results are obtained by processing using the Logits function.
D, a basic knowledge graph is updated according to the predicted learning preferences, and a personalized knowledge graph of all courses conforming to learning conditions of students is generated.
E, a personalized knowledge graph of all courses that students need to learn in the current academic year is collected to generate a personalized knowledge base exclusive to each individual.

Preferably, in Step 1, realizing the personalized knowledge graph merging of all courses includes the following steps:
1.1. extracting knowledge points and structure information of all personalized knowledge graphs of students in the current academic year, wherein the index table includes knowledge points and relationships;
1.2. using a knowledge point representation learning method for representation learning according to the index table;
1.3. calculating the similarity between the knowledge point and the relationship according to the index table to perform tasks of knowledge point alignment and link prediction;
1.4. automatically generating a personalized knowledge graph of multi-course merging.

Preferably, a step of designing a structure of the index table is as follows:
1.1.1. numbering documents of a plurality of teaching materials;
1.1.2. taking key words of knowledge points in the knowledge graph as key values;
1.1.3. recording the space occupied by knowledge points in four types: basic concept explanation, application explanation, intensive explanation and testing;
1.1.4. a document frequency p, wherein a frequency of knowledge points appearing in a document collection is recorded;
1.1.5. associated document ID;
1.1.6. a lexical term frequency q, wherein a frequency of key words appearing in a specific document is recorded;
1.1.7. position information, wherein the specific position information of key words in the document is recorded;
1.1.8. relationship information, which is used to describe the connection and association between knowledge points.

Preferably, a step of designing knowledge point representation learning is as follows:
1.2.1. learning an initial embedding vector of an entity and a relationship by using a TransR model: training the TransR model by minimizing conversion errors among a head entity, a relationship and a tail entity to obtain the initial embedding vectors of each entity and relationship;
1.2.2. determining a plurality of different levels of embedding vectors according to task requirements: setting two levels of embedding vectors, in which one lower dimension is used to represent surface semantics, and the other higher dimension is used to represent deeper semantics;
1.2.3. for each entity and relationship, merging the plurality of learned embedding vectors, and connecting the plurality of embedding vectors to form a higher-dimensional embedding representation to obtain the TransR model with multi-level embedding;
wherein the surface semantics is represented by one dimension, the deeper semantics is greater than one dimension, such as two dimensions, and the higher semantics is greater than two dimensions, such as three dimensions;
1.2.4. using the TransR model with multi-level embedding obtained in Step 1.2.3 for joint training: in the training process, using relation triplets, entity attribute information of the knowledge graph and context information of the knowledge graph to assist in learning more accurate embedding representation;
1.2.5. iterating the training process, constantly optimizing parameters of multi-level embedding and the TransR model with multi-level embedding, and obtaining a final entity embedding vector and a relationship embedding vector; wherein the relationship includes one or more factors of an entity attribute value, relationship information and semantic association.

Preferably, a process step of entity alignment is as follows:
1a. calculating the similarity between the obtained entity embedding vector and the relationship embedding vector, representing the position of the entity in the semantic space by the output entity embedding vector, the vectors usually having fixed dimensions, capturing the semantic similarity between entities, and performing the entity alignment task by calculating the similarity f(s) between entity vectors;
1b. capturing the semantic differences between different relationships by the output relationship embedding vector, and performing the link prediction task by calculating the similarity f(g) between the relationship vectors to obtain the personalized knowledge graph of multi-course merging.

Preferably, a similarity calculation method includes cosine similarity, Euclidean distance and Mahalanobis distance.

Preferably, the evaluation method of Step 3 is as follows:
evaluating the generated learning path by using an accuracy index of link prediction, in which the link prediction accuracy is:

$$ACC = \frac{P\_r}{R\_r}$$

where P_r denotes the link prediction result; and R_r denotes the real link result, ACC∈[0, 1].

The present disclosure further provides a system of generating a personalized learning path based on multi-course knowledge graph merging, wherein the system includes a personalized knowledge graph base module, a multi-course knowledge graph merging module, a path generation module and a path evaluation module; the system forms a response to external events by defining a finite state, and a response mechanism is as follows:
a user logs into the system, the system state changes from Init state to Log_in state at this time, and the system enters the personalized knowledge graph base module; the system performs information matching according to the login information of the user, the system state changes from Log_in state to Match state at this time until the personalized knowledge graph of all courses belonging to the user is found; thereafter, the system state changes from Match state to Merge state, the system enters the multi-course knowledge graph merging module, and the personalized knowledge graphs of all courses are merged; the system state changes from Merge state to Path_g state after merging is completed, the system enters the path generation module, and the subsequent learning path is generated according to the user's own situation; the system state changes from Path_g state to Path_e state after the learning path is generated, the system enters the path evaluation module, and the generated learning path is evaluated by using the accuracy index of link prediction.

Preferably, the finite state and the conversion rule of the system are designed as follows:
(1) Init state: the system is in the initial state at this time;
(2) Log_in state: this is the login state, in which a user logs into the system, the system state changes from Init state to Log_in state at this time, and the system enters the personalized knowledge graph base module;
(3) Match state: this is the information matching state, in which the system performs information matching according to the login information of the user, and the system state changes from Log_in state to Match state at this time until the personalized knowledge graph of all courses belonging to the user is found;
(4) Merge state: this is the merging state, in which after the personalized knowledge graph of all courses belonging to the user is obtained, the system state changes from Match state to Merge state, the system enters the multi-course knowledge graph merging module, and the personalized knowledge graphs of all courses are merged;
(5) Path_g state: this is the path generation state, in which the system state changes from Merge state to Path_g state after merging is completed, the system enters the path generation module, and the subsequent learning path is generated according to the user's own situation;
(6) Path_e state: this is the path evaluation state, in which the system state changes from Path_g state to Path_e state after the learning path is generated, the system enters the path evaluation module, and the generated learning path is evaluated by using the accuracy index of link prediction;
(7) End state: this is the end state, in which the system state changes from Path_e state to End state after the evaluation and optimization are completed, and the multi-course learning path generation task is completed.

The present disclosure has the following advantages. An entity is mapped to a low-dimensional embedding space by an entity representation learning method. The semantic information and the potential relationship of the entity are expressed in the form of continuous vectors. This representation can support entity similarity calculation and entity matching, so that tasks of entity alignment and link prediction can be performed in a multi-course knowledge graph, which assists in eliminating entity duplication, filling knowledge gaps and improving the consistency and integrity of knowledge.

### BRIEF DESCRIPTION OF THE DRAWINGS

FIG. 1 is an overall flow chart of the present disclosure.

FIG. 2 is a schematic diagram of converting the finite state according to the present disclosure.

FIG. 3 is a schematic diagram of designing a structure of an index table according to the present disclosure.

FIG. 4 is a schematic diagram of a method of merging a multi-course knowledge graph according to the present disclosure.

FIG. 5 is a schematic diagram of a method of generating and evaluating a personalized learning path according to the present disclosure.

### DETAILED DESCRIPTION OF THE EMBODIMENTS

The embodiments of the present disclosure will be described with reference to specific embodiments hereinafter. Those familiar with this technology can easily understand other advantages and effects of the present disclosure from the contents disclosed in this specification. Obviously, the described embodiments are some of the embodiments of the present disclosure, rather than all of the embodiments. Based on the embodiments in the present disclosure, all other embodiments obtained by those skilled in the art without paying creative labor belong to the scope of protection of the present disclosure.

The present disclosure provides a method of generating a personalized learning path based on multi-course knowledge graph merging, wherein the method includes constructing a personalized knowledge base to obtain the personalized knowledge graph of all courses that students need to learn in the current academic year.

A step of constructing a personalized knowledge base is as follows:
A, collecting physiological data signals of students, including electroencephalogram signals, electrocardiogram signals and video signals, through a head-mounted electroencephalogram device, a patch electrocardiogram device and a suspended monitoring device;
B, extracting key features from the collected physiological data signals to obtain key data features of students, including electroencephalogram, electrocardiogram, body temperature, posture and facial expression features;
C, designing a multimodal physiological data merging mining method to perform data mining and predict learning preferences of students;
D, updating a basic knowledge graph according to the predicted learning preferences, and generating a personalized knowledge graph of all courses conforming to learning conditions of students;
E, collecting a personalized knowledge graph of all courses that students need to learn in the current academic year to generate a personalized knowledge base exclusive to each individual;

The method of generating a personalized learning path based on multi-course knowledge graph merging further includes the following steps.

Step 1: an entity alignment technology is used to realize the personalized knowledge graph merging of all courses, and a personalized knowledge graph of multi-course merging is automatically generated by establishing an index table, which specifically includes the following steps.
(1.1) knowledge points and structure information of all personalized knowledge graphs of students in the current academic year are extracted, wherein the index table includes knowledge points and relationships.

A step of designing a structure of the index table is as follows:
(1.1.1) numbering documents of a plurality of teaching materials;
(1.1.2) taking key words of knowledge points in the knowledge graph as key values;
(1.1.3) recording the space occupied by knowledge points in four types: basic concept explanation, application explanation, intensive explanation and testing;
(1.1.4) a document frequency p, wherein a frequency of knowledge points appearing in a document collection is recorded;
(1.1.5) associated document ID;
(1.1.6) a lexical term frequency q, wherein a frequency of key words appearing in a specific document is recorded;
(1.1.7) position information, wherein the specific position information of key words in the document is recorded;
(1.1.8) relationship information, which is used to describe the connection and association between knowledge points.
(1.2) a knowledge point representation learning method is used for representation learning according to the index table.

A step of designing knowledge point representation learning is as follows:
(1.2.1) learning an initial embedding vector of an entity and a relationship by using a TransR model: training the TransR model by minimizing conversion errors among a head entity, a relationship and a tail entity to obtain the initial embedding vectors of each entity and relationship;
(1.2.2) determining a plurality of different levels of embedding vectors according to task requirements: setting two levels of embedding vectors, in which one lower dimension is used to represent surface semantics, and the other higher dimension is used to represent deeper semantics;
(1.2.3) for each entity and relationship, merging the plurality of learned embedding vectors, and connecting the plurality of embedding vectors to form a higher-dimensional embedding representation to obtain the TransR model with multi-level embedding;
(1.2.4) using the TransR model with multi-level embedding obtained in Step (1.2.3) for joint training: in the training process, using relation triplets, entity attribute information of the knowledge graph and context information of the knowledge graph to assist in learning more accurate embedding representation;
(1.2.5) iterating the training process, constantly optimizing parameters of multi-level embedding and the TransR model with multi-level embedding, and obtaining a final entity embedding vector and a relationship embedding vector; wherein the relationship includes one or more factors of an entity attribute value, relationship information and semantic association.
(1.3) the similarity between the knowledge point and the relationship is calculated according to the index table to perform tasks of knowledge point alignment and link prediction.

A process step of entity alignment is as follows:
1a. calculating the similarity between the obtained entity embedding vector and the relationship embedding vector, representing the position of the entity in the semantic space by the output entity embedding vector, the vectors usually having fixed dimensions, capturing the semantic similarity between entities, and performing the entity alignment task by calculating the similarity between entity vectors;
1b. capturing the semantic differences between different relationships by the output relationship embedding vector, and performing the link prediction task by calculating the similarity between the relationship vectors to obtain the personalized knowledge graph of multi-course merging.

A commonly used similarity calculation method includes cosine similarity, Euclidean distance and Mahalanobis distance.
(1.4) a personalized knowledge graph of multi-course merging is automatically generated.

Step 2, a subsequent learning path is generated according to a current learning progress of users and the personalized knowledge graph of multi-course merging.

Step 3: the generated learning path is evaluated in terms of link prediction accuracy.

Preferably, the path evaluation method is designed as follows:
evaluating the generated learning path by using an accuracy index of link prediction, in which the link prediction accuracy is:

$$ACC = \frac{P\_r}{R\_r}$$

where P_r denotes the link prediction result; and R_r denotes the real link result, ACC∈[0, 1].

### Embodiment 2

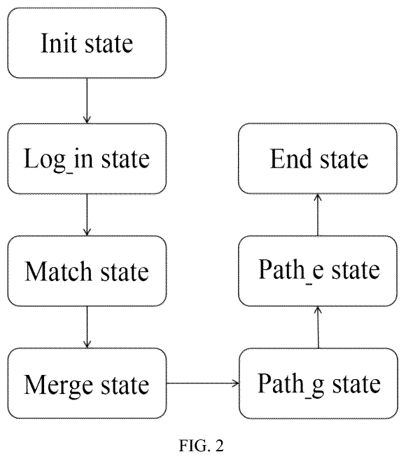

The present disclosure further provides a system of generating a personalized learning path based on multi-course knowledge graph merging, wherein the system includes a personalized knowledge graph base module, a multi-course knowledge graph merging module, a path generation module and a path evaluation module. As shown in FIG. 2, the system designed in this embodiment forms a response to external events by defining a finite state, and a response mechanism is as follows:
a user logs into the system, the system state changes from Init state to Log_in state at this time, and the system enters the personalized knowledge graph base module; the system performs information matching according to the login information of the user, the system state changes from Log_in state to Match state at this time until the personalized knowledge graph of all courses belonging to the user is found; thereafter, the system state changes from Match state to Merge state, the system enters the multi-course knowledge graph merging module, and the personalized knowledge graphs of all courses are merged; the system state changes from Merge state to Path_g state after merging is completed, the system enters the path generation module, and the subsequent learning path is generated according to the user's own situation; the system state changes from Path_g state to Path_e state after the learning path is generated, the system enters the path evaluation module, and the generated learning path is evaluated by using the accuracy index of link prediction.

The finite state and the conversion rule of the system are designed as follows:
(1) Init state: the system is in the initial state at this time;
(2) Log_in state: this is the login state, in which a user logs into the system, the system state changes from Init state to Log_in state at this time, and the system enters the personalized knowledge graph base module;
(3) Match state: this is the information matching state, in which the system performs information matching according to the login information of the user, and the system state changes from Log_in state to Match state at this time until the personalized knowledge graph of all courses belonging to the user is found;
(4) Merge state: this is the merging state, in which after the personalized knowledge graph of all courses belonging to the user is obtained, the system state changes from Match state to Merge state, the system enters the multi-course knowledge graph merging module, and the personalized knowledge graphs of all courses are merged;
(5) Path_g state: this is the path generation state, in which the system state changes from Merge state to Path_g state after merging is completed, the system enters the path generation module, and the subsequent learning path is generated according to the user's own situation;
(6) Path_e state: this is the path evaluation state, in which the system state changes from Path_g state to Path_e state after the learning path is generated, the system enters the path evaluation module, and the generated learning path is evaluated by using the accuracy index of link prediction;
(7) End state: this is the end state, in which the system state changes from Path_e state to End state after the evaluation and optimization are completed, and the multi-course learning path generation task is completed.

### Embodiment 3

The following case illustrates how to apply the system of generating the personalized learning path based on multi-course knowledge graph merging.

In order to improve the universality of the case, a representative, abstract and universal catalogue item is used to describe the teaching materials.

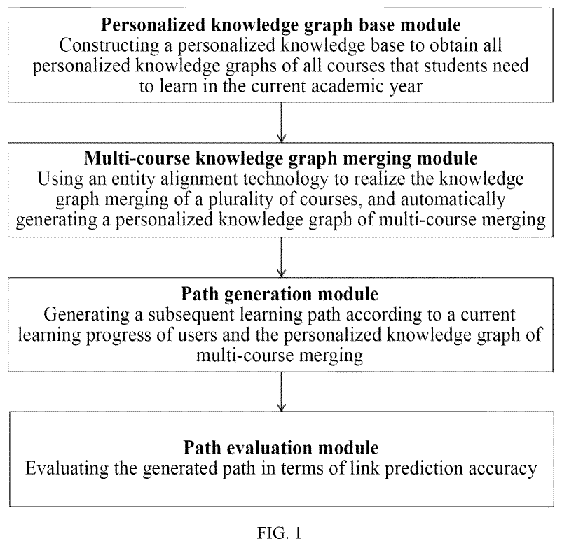

As shown in FIG. 1, the system of generating the personalized learning path based on multi-course knowledge graph merging includes a personalized knowledge graph base module, a multi-course knowledge graph merging module, a path generation module and a path evaluation module.

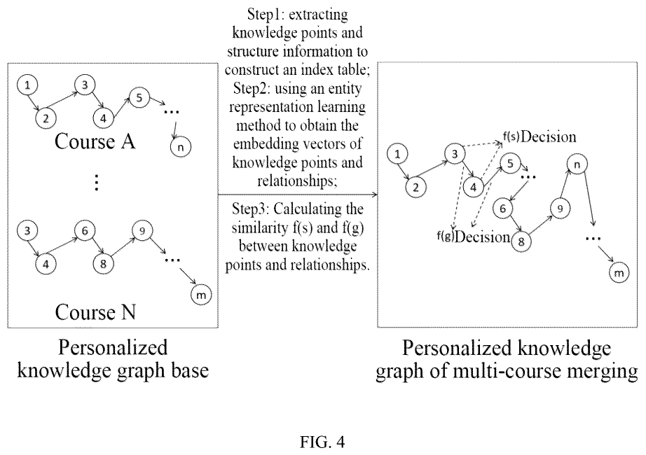

The personalized knowledge graph base module is configured to construct a personalized knowledge base to obtain all personalized knowledge graphs of courses A to N that students need to learn in the current academic year, such as the personalized knowledge graph base as shown in FIG. 4. The multi-course knowledge graph merging module is configured to use an entity alignment technology to realize the personalized knowledge graph merging of courses A to N. The path generation module is configured to generate a subsequent learning path according to a current learning progress of users. The path evaluation module is configured to evaluate the generated path in terms of link prediction accuracy.

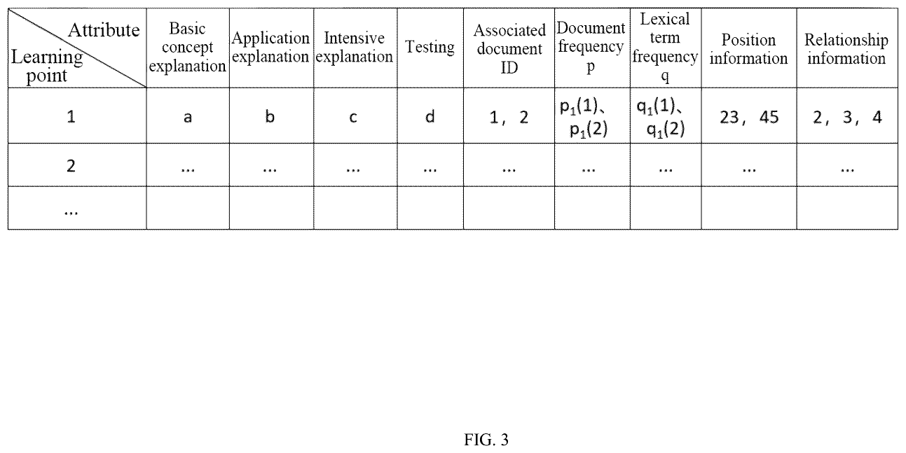

First, an index table as shown in FIG. 3 is constructed according to the personalized knowledge graph of courses A to N.

In the index table, a, b, c and d represent the space proportion of current knowledge points in four parts: basic concept, application, intensification and testing; 1, 2, . . . , n represent the document ID of courses A to N, where 1, 2 represent document 1 and document 2 related to knowledge point 1; p represents the document frequency, wherein a frequency of knowledge points appearing in a document collection is recorded; q represents a lexical term frequency, wherein a frequency of key words appearing in a specific document is recorded; position information is shown, wherein the specific position information of key words in the document is recorded, and knowledge point 1 appears on page 23 of document 1 and page 45 of document 2; relationship information is used to describe the connection and association between knowledge points, wherein knowledge point 1 is associated with knowledge points 2, 3 and 4, and the association here includes semantic association and context association.

Thereafter, as shown in FIG. 4, knowledge point representation learning is used to represent the embedded vectors of the entity and the relationship according to the contents of the index table. The similarity between the obtained entity embedding vector and the relationship embedding vector is calculated, and the entity alignment task is performed by calculating the similarity f(s) between the entity vectors. The link prediction task is performed by calculating the similarity f(g) between the relationship vectors.

If the knowledge points 3 and 4 of courses A and N are similar through the calculation of the above process, entity alignment is performed to remove redundant knowledge points, and then the relationship between knowledge points is re-linked according to the calculated relationship similarity. If the relationship between knowledge point 4 and knowledge point 5 is more similar, knowledge point 5 is linked. Similarly, the link of each pair of knowledge points is obtained to automatically generate the personalized knowledge graph of multi-course merging.

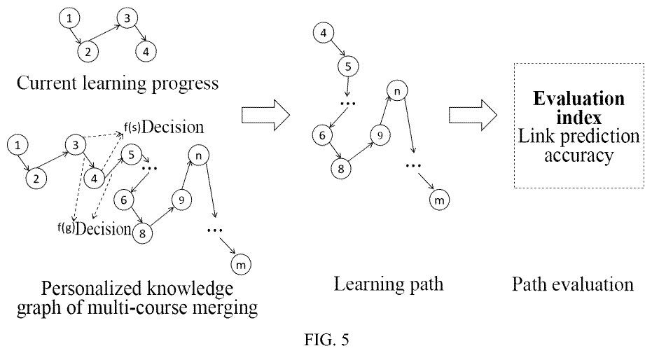

Finally, as shown in FIG. 5, in conjunction with the current learning progress and the personalized knowledge graph of multi-course merging, the next learning path of students is given to complete multi-course link learning. The generated learning path is evaluated by using the accuracy index of link prediction at the same time.

Although the present disclosure has been described in detail with reference to general description and specific embodiments, it is obvious to those skilled in the art that some modifications or improvements can be made on the basis of the present disclosure. Therefore, these modifications or improvements made without departing from the spirit of the present disclosure belong to the scope of protection of the present disclosure.

What is claimed is:

1. A method of generating a personalized learning path based on multi-course knowledge graph merging, wherein the method comprises constructing a personalized knowledge base, wherein a step of constructing a personalized knowledge base is as follows:
A, collecting physiological data signals of students, comprising electroencephalogram signals, electrocardiogram signals and video signals, through a head-mounted electroencephalogram device, a patch electrocardiogram device and a suspended monitoring device;
B, extracting key features from the collected physiological data signals to obtain key data features of students, comprising electroencephalogram, electrocardiogram, body temperature, posture and facial expression features;
C, designing a multimodal physiological data merging mining method to perform data mining and predict learning preferences of students;
D, updating a basic knowledge graph according to the predicted learning preferences, and generating a personalized knowledge graph of all courses conforming to learning conditions of students;
E, collecting a personalized knowledge graph of all courses that students need to learn in the current academic year to generate a personalized knowledge base exclusive to each individual;
wherein obtaining the personalized knowledge graph of all courses that students need to learn in the current academic year further comprises the following steps:
Step 1: using an entity alignment technology to realize the personalized knowledge graph merging of all courses, and automatically generating a personalized knowledge graph of multi-course merging by establishing an index table;
wherein realizing the personalized knowledge graph merging of all courses comprises the following steps:
1.1. extracting knowledge points and structure information of all personalized knowledge graphs of students in the current academic year, wherein the index table comprises knowledge points and relationships;
wherein a step of designing a structure of the index table is as follows:
1.1.1. numbering documents of a plurality of teaching materials;
1.1.2. taking key words of knowledge points in the knowledge graph as key values;
1.1.3. recording the space occupied by knowledge points in four types: basic concept explanation, application explanation, intensive explanation and testing;
1.1.4. a document frequency, wherein a frequency of knowledge points appearing in a document collection is recorded;
1.1.5. associated document ID;
1.1.6. a lexical term frequency, wherein a frequency of key words appearing in a specific document is recorded;
1.1.7. position information, wherein the specific position information of key words in the document is recorded;
1.1.8. relationship information, which is used to describe the connection and association between knowledge points;
1.2. using a knowledge point representation learning method for representation learning according to the index table;
wherein a step of designing knowledge point representation learning is as follows:
1.2.1. learning an initial embedding vector of an entity and a relationship by using a TransR model: training the TransR model by minimizing conversion errors among a head entity, a relationship and a tail entity to obtain the initial embedding vectors of each entity and relationship;
1.2.2. determining a plurality of different levels of embedding vectors according to task requirements: setting two levels of embedding vectors, in which one dimension is used to represent surface semantics, and the other dimension is used to represent deeper semantics;
1.2.3. for each entity and relationship, merging the plurality of learned embedding vectors, and connecting the plurality of embedding vectors to form a higher-dimensional embedding representation to obtain the TransR model with multi-level embedding;
1.2.4. using the TransR model with multi-level embedding obtained in Step 1.2.3 for joint training: in the training process, using relation triplets, entity attribute information of the knowledge graph and context information of the knowledge graph to assist in learning more accurate embedding representation;
1.2.5. iterating the training process, constantly optimizing parameters of multi-level embedding and the TransR model with multi-level embedding, and obtaining a final entity embedding vector and a relationship embedding vector; wherein the relationship comprises one or more factors of an entity attribute value, relationship information and semantic association;
1.3. calculating the similarity between the knowledge point and the relationship according to the index table to perform tasks of knowledge point alignment and link prediction;
1.4. automatically generating a personalized knowledge graph of multi-course merging;
wherein a process step of entity alignment is as follows:
1a. calculating the similarity between the obtained entity embedding vector and the relationship embedding vector, representing the position of the entity in the semantic space by the output entity embedding vector, capturing the semantic similarity between entities, and performing the entity alignment task by calculating the similarity between entity vectors;
1b. capturing the semantic differences between different relationships by the output relationship embedding vector, and performing the link prediction task by calculating the similarity between the relationship vectors to obtain the personalized knowledge graph of multi-course merging;
wherein a similarity calculation method comprises cosine similarity, Euclidean distance and Mahalanobis distance;
Step 2, generating a subsequent learning path according to a current learning progress of users and the personalized knowledge graph of multi-course merging;
Step 3: evaluating the generated learning path in terms of link prediction accuracy;
wherein the evaluation method of Step 3 is as follows:
evaluating the generated learning path by using an accuracy index of link prediction, in which the link prediction accuracy is:
$$ACC = \frac{P\_r}{R\_r}$$
where P_r denotes the link prediction result; and R_r denotes the real link result, ACC∈[0,1].

2. A system of realizing the method according to claim 1, wherein the system comprises a personalized knowledge graph base module, a multi-course knowledge graph merging module, a path generation module and a path evaluation module; the system forms a response to external events by defining a finite state, and a response mechanism is as follows:
a user logs into the system, the system state changes from Init state to Log_in state at this time, and the system enters the personalized knowledge graph base module; the system performs information matching according to the login information of the user, the system state changes from Log_in state to Match state at this time until the personalized knowledge graph of all courses belonging to the user is found; thereafter, the system state changes from Match state to Merge state, the system enters the multi-course knowledge graph merging module, and the personalized knowledge graphs of all courses are merged; the system state changes from Merge state to Path_g state after merging is completed, the system enters the path generation module, and the subsequent learning path is generated according to the user's own situation; the system state changes from Path_g state to Path_e state after the learning path is generated, the system enters the path evaluation module, and the generated learning path is evaluated by using the accuracy index of link prediction.

3. The system according to claim 2, wherein the finite state and the conversion rule of the system are designed as follows:
(1) Init state: the system is in the initial state at this time;
(2) Log_in state: this is the login state, in which a user logs into the system, the system state changes from Init state to Log_in state at this time, and the system enters the personalized knowledge graph base module;
(3) Match state: this is the information matching state, in which the system performs information matching according to the login information of the user, and the system state changes from Log_in state to Match state at this time until the personalized knowledge graph of all courses belonging to the user is found;
(4) Merge state: this is the merging state, in which after the personalized knowledge graph of all courses belonging to the user is obtained, the system state changes from Match state to Merge state, the system enters the multi-course knowledge graph merging module, and the personalized knowledge graphs of all courses are merged;
(5) Path_g state: this is the path generation state, in which the system state changes from Merge state to Path_g state after merging is completed, the system enters the path generation module, and the subsequent learning path is generated according to the user's own situation;
(6) Path_e state: this is the path evaluation state, in which the system state changes from Path_g state to Path_e state after the learning path is generated, the system enters the path evaluation module, and the generated learning path is evaluated by using the accuracy index of link prediction;
(7) End state: this is the end state, in which the system state changes from Path_e state to End state after the evaluation and optimization are completed, and the multi-course learning path generation task is completed.
